(19) United States
(12) Patent Application Publication
(10) Pub. No.: US 2008/0125477 A1
(43) Pub. Date: May 29, 2008

(54) 7-(ACRYLOYL) INDOLE COMPOSITIONS AND METHODS OF MAKING AND USING SAME

(75) Inventors: Jasbir Singh, Naperville, IL (US); Matt Shaoming Duan, Foster City, CA (US); Thorsteinn Thorsteinsson, Greensboro, NC (US); Mitchell B. Friedman, Buffalo Grove, IL (US); Mark Gurney, Grand Rapids, MI (US)

Correspondence Address: HESLIN ROTHENBERG FARLEY & MESITI PC, 5 COLUMBIA CIRCLE, ALBANY, NY, 12203, US

(73) Assignee: DECODE GENETICS, EHF., Reykjavik (IS)

(21) Appl. No.: 11/748,897

(22) Filed: May 15, 2007

Related U.S. Application Data
(60) Provisional application No. 60/800,806, filed on May 16, 2006.

(51) Int. Cl. A61K 31/404; A61P 31/00; A61P 19/00; A61P 3/00; A61P 15/00; A61P 25/00; A61P 11/00

(52) U.S. Cl. 514/414

(57) ABSTRACT

The present invention is directed to pharmaceutical compositions of 7-(acryloyl)indoles, such as 4,5 -Dichloro-thiophene-2-sulfonic acid [(E)-3-[1-(2,4-dichlorophenylmethyl)-5-fluoro-3-methyl-1H-indol-7-yl]-acryloyl]amide (DTSI), having a structure shown below.

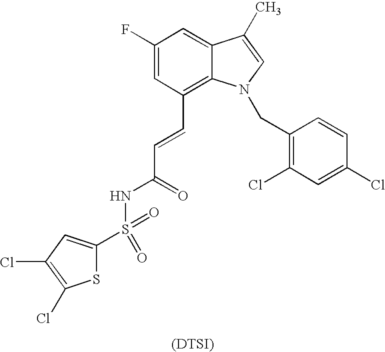

The invention is also directed to methods of treatment utilizing formulations of the DTSI and processes of preparation of the formulations.

### CROSS-REFERENCE TO RELATED APPLICATIONS

This application claims priority to U.S. Provisional Application No. 60/800,806 filed on May 16, 2006, which is incorporated herein by reference in its entirety.

### FIELD OF THE INVENTION

The present invention relates to pharmaceutical compositions of 7-(acryloyl) indoles, such as 4,5-dichlorothiophene-2-sulfonic acid [(E)-3-[1-(2,4-dichlorophenylmethyl)-5-fluoro-3-methyl-1H-indol-7-yl]-acryloyl]amide, processes for preparation of such compositions and their methods of use.

### BACKGROUND OF THE INVENTION

Atherosclerosis is the pathology underlying several of mankind's most lethal diseases, such as myocardial infarction and peripheral arterial occlusive disease (PAOD). PAOD represents atherosclerosis of the large and medium arteries of the limbs, particularly to the lower extremities, and includes the aorta and iliac arteries. It often coexists with coronary artery disease and cerebrovascular disease. Persons with PAOD are at increased risk of other vascular events such as myocardial infarction or stroke.

Ortho-substituted phenyl acylsulfonamides and their utility for treating prostaglandin-mediated disorders are described in U.S. Pat. No. 6,242,493 and in two articles by Juteau et al. [BioOrg. Med. Chem. 9, 1977-1984 (2001)] and Gallant et al. [BioOrg. Med. Chem. Let. 12, 2583-2586 (2002)], the disclosures of which are incorporated herein by reference.

A promising new candidate for treating PAOD is 4,5-dichlorothiophene-2-sulfonic acid [(E)-3-[1-(2,4-dichlorophenylmethyl)-5-fluoro-3-methyl-1H-indol-7-yl]-acryloyl]amide (hereafter, “DTSI”), which is disclosed in U.S. application Ser. No. 11/169,161 and the corresponding PCT/US05/23009, both filed Jun. 27, 2005 and both incorporated herein by reference for their disclosures of the synthesis and activity of DTSI. However, DTSI exhibits poor aqueous solubility. This presents problems for preparation of suitable formulations. Additionally, poor aqueous solubility presents a problem of inadequate drug bioavailability.

### SUMMARY OF THE INVENTION

The present invention is directed to overcoming the above-mentioned problems by providing novel pharmaceutical compositions of DTSI. The chemical structure of DTSI is shown below.

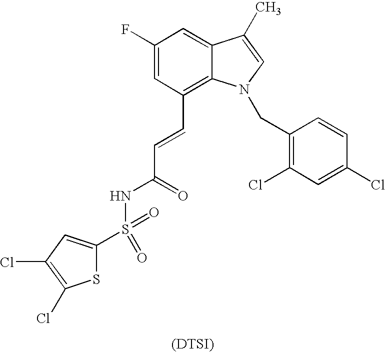

Thus, it is an object of the present invention to provide pharmaceutical compositions comprising DTSI, or a pharmaceutically acceptable salt thereof, and at least one pharmaceutically acceptable excipient. In one embodiment, the composition is a solid single-phase composition.

The invention is also directed to methods of treatment utilizing presently disclosed formulations of DTSI. Furthermore, the present invention provides processes of preparation of the described compositions. One such process is a process for preparation of a solid oral pharmaceutical in unit dosage form, said process comprising: a) mixing DTSI, or a pharmaceutically acceptable salt thereof, and at least one pharmaceutically acceptable fusible excipient; b) subjecting the mixture to injection molding or extrusion and c) processing the mixture into an oral unit dosage form.

### DETAILED DESCRIPTION OF THE INVENTION

The present invention provides pharmaceutical compositions comprising DTSI

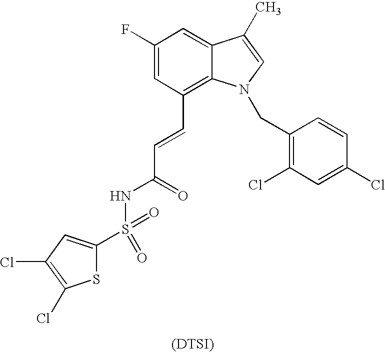

or a pharmaceutically acceptable salt thereof, and at least one pharmaceutically acceptable excipient.

DTSI is a potent, selective EP3 receptor antagonist, as demonstrated by the data presented in the Experimental section. A process for preparation of DTSI is also provided in the Experimental section.

The term “pharmaceutically acceptable salts” embraces salts of DTSI with pharmaceutically acceptable bases. Suitable pharmaceutically acceptable base addition salts for the compounds of the present invention include, but are not limited to, metallic salts made from aluminum calcium, lithium, magnesium, potassium, sodium and zinc or organic salts made from lysine, arginine, N,N′-dibenzylethylenediamine, chloroprocaine, ethylenediamine, meglumine (N-methylglucamine) and procaine.

In one embodiment, the pharmaceutically acceptable salt is chosen from a salt with a pharmaceutically acceptable primary, secondary, or tertiary amine compound, and a pharmaceutically acceptable quaternary ammonium compound. Examples of such salts are salts of DTSI with choline, choline phosphate, betaine, sarcosine, N,N-dimethyl glycine, tromethamine, ethanolamine, diethanolamine, or triethanolamine.

In one embodiment, the composition is a solid, single-phase composition. Such composition may be described as a substantially amorphous solid solution, which is a homogenous solution of DTSI in at least one excipient. The term “substantially amorphous” as applied to solid solutions as used herein means that the solid solutions as measured by X-ray diffraction analysis are greater than 90% amorphous and such solid solutions are homogenous and consist of a single phase.

The solid, single-phase composition may be prepared by a well known process of hot-melt extrusion. A review of hot-melt extrusion processes is provided, for example, in Chokshi et al., Iranian Journal of Pharmaceutical Research, 3: 3-16 (2004).

Surprisingly, it has been found that solid, single-phase formulations of the DTSI have superior pharmacokinetic parameters. As already mentioned, DTSI exhibits poor aqueous solubility, which presents formulation problems when dosages on the milligram scale are contemplated. However, even though some PEG3350 and LABRASOL® (glyceryl caprylate/caprate and polyethylene glycol caprylate/caprate complex) derived formulations showed superior pharmacokinetic profile (higher Cₘₐₓ and AUC) when compared to the micronized powder, based on the solubility profile in these formulations, one would need multiple capsules for dosing in the 100 mg range. Formulations that retain or provide further improved pharmacokinetic profile while allowing one to significantly improve the active ingredient load are advantageous. As described below, hot-melt solid single-phase compositions exhibit unexpectedly superior pharmacokinetic properties.

For the majority of tested formulations, the dissolution of DTSI (active pharmaceutical ingredient, API) in excipient to provide a homogenous solution was achieved around 65° C. and these typically provided API solubility of ≦70 mg/g. However, for several excipient combinations, in particular those that contained MYRJ (poloxyethylene stearate) or TPGS (Vitamin E TPGS, which is chemically, d-α-tocopheryl polyethylene glycol succinate) as one of the excipients, higher concentration of dissolved API could be achieved by using a higher bath temperature of up to 90° C. and using sonication or stirring.

Based on the rat pharmacokinetic studies, the best two TPGS/PEG3350-derived formulations were TPGS/PEG 3350 (1/1) and PEG 3350/TPGS (25/75). These were tested in dogs. The obtained data surprisingly showed that while the two hot-melt formulations and micronized powder formulations provided similar AUC, the two hot-melt formulations provided significantly higher Cₘₐₓ.

Furthermore, initial studies indicate that hot-melt formulations that include small amounts of a polymer such as hydroxypropyl methylcellulose (HPMC), allow preparation of hot soluble formulations which, upon cooling, can provide homogeneous solid formulation with significantly high API load per gram of the formulated solid. One such solid formulation contains a suspension of API and Vitamin E TPGS (50/50 mixture of API in Vitamin E TPGS) and a small amount of HPMC. Under visual observations, this forms a uniform solution that solidifies uniformly.

In one embodiment, the at least one pharmaceutically acceptable excipient is chosen from Vitamin E TPGS, polyethylene glycol (PEG), and combinations thereof. In another embodiment, the at least one pharmaceutically acceptable excipient is chosen from Vitamin E TPGS, polyethylene glycol, hydroxypropyl methylcellulose, and combinations thereof. In yet another embodiment, the at least one pharmaceutically acceptable excipient is chosen from Vitamin E TPGS, polyethylene glycol, hydroxypropyl methylcellulose, polyglycolyzed glyceride, polyoxyethylene glycol ester, polyoxyethylene sorbitan fatty acid ester, choline, and combinations thereof. Choline may be used to form a salt with DTSI and/or as a pharmaceutically acceptable excipient.

In one preferred embodiment the ratio of Vitamin E TPGS to PEG3350 is about 1:1. In another preferred embodiment, the ratio of Vitamin E TPGS to PEG3350 is about 3:1. In another embodiment, the ratio of Vitamin E TPGS to PEG3350 is about 2:1. In another embodiment, the ratio of Vitamin E TPGS to PEG3350 is in the range of from about 1:4 to about 4:1. In another embodiment, the ratio of Vitamin E TPGS to PEG3350 is about 1:9. In another embodiment, the ratio of Vitamin E TPGS to PEG3350 is about 1:4. In another embodiment, the ratio of Vitamin E TPGS to PEG3350 is about 3:7. In another embodiment, the ratio of Vitamin E TPGS to PEG3350 is about 2:3. In another embodiment, the ratio of Vitamin E TPGS to PEG3350 is about 1:1. In another embodiment, the ratio of Vitamin E TPGS to PEG3350 is about 3:2. In another embodiment, the ratio of Vitamin E TPGS to PEG3350 is about 7:3. In another embodiment, the ratio of Vitamin E TPGS to PEG3350 is about 4:1. In another embodiment, the ratio of Vitamin E TPGS to PEG3350 is about 9:1.

The ratio of DTSI to excipient or excipients may be in the range from about 1:100 to about 1:1. In another embodiment the ratio of DTSI to excipient or excipients may be in the range from about 1:20 to about 1:1. In yet another embodiment the ratio of DTSI to excipient or excipients may be in the range from about 1:15 to about 1:5.

In one embodiment, the ratio of DTSI to excipient or excipients is about 1:90. In another embodiment, the ratio of DTSI to excipient or excipients is about 1:80. In another embodiment, the ratio of DTSI to excipient or excipients is about 1:70. In another embodiment, the ratio of DTSI to excipient or excipients is about 1:60. In another embodiment, the ratio of DTSI to excipient or excipients is about 1:50. In another embodiment, the ratio of DTSI to excipient or excipients is about 1:40. In another embodiment, the ratio of DTSI to excipient or excipients is about 1:30. In another embodiment, the ratio of DTSI to excipient or excipients is about 1:20. In another embodiment, the ratio of DTSI to excipient or excipients is about 1:15. In another embodiment, the ratio of DTSI to excipient or excipients is about 1:14. In another embodiment, the ratio of DTSI to excipient or excipients is about 1:13. In another embodiment, the ratio of DTSI to excipient or excipients is about 1:12. In another embodiment, the ratio of DTSI to excipient or excipients is about 1:11. In another embodiment, the ratio of DTSI to excipient or excipients is about 1:10. In another embodiment, the ratio of DTSI to excipient or excipients is about 1:9. In another embodiment, the ratio of DTSI to excipient or excipients is about 1:8. In another embodiment, the ratio of DTSI to excipient or excipients is about 1:7. In another embodiment, the ratio of DTSI to excipient or excipients is about 1:6. In another embodiment, the ratio of DTSI to excipient or excipients is about 1:5. In another embodiment, the ratio of DTSI to excipient or excipients is about 1:4. In another embodiment, the ratio of DTSI to excipient or excipients is about 3:7. In another embodiment, the ratio of DTSI to excipient or excipients is about 2:3. In another embodiment, the ratio of DTSI to excipient or excipients is about 1:1.

In the above-described embodiments, polyglycolyzed glyceride may be a glyceryl caprylate/caprate and polyethylene glycol caprylate/caprate complex, such as commercially available LABRASOL®. Polyoxyethylene glycol ester may be chosen from poloxyethylene 8 stearate (MYRJ 45), poloxyethylene 40 stearate (MYRJ 52), polyoxyethylene 100 stearate (MYRJ 59), and combinations thereof. Polyoxyethylene sorbitan fatty acid ester may be polyoxyethylene 20 sorbitan monooleate (TWEEN 80®).

To facilitate self-emulsification, pharmaceutically acceptable surfactants can optionally be used in the composition, which include, for example, polyoxyl castor oils (e.g., Cremophor® RH40, Cremophor® EL), polyoxyl hydrogenated castor oils, polysorbates (e.g., Tween 80® ), peglicol 6-oleate, polyoxyethylene stearates, polyglycolyzed glycerides (e.g., GELUCIRE 44/14), poloxamers (e.g., PLURONIC F68), sodium lauryl sulfate, and mixtures thereof. Vitamin E TPGS, alone or in combination, may also function as a surfactant.

It has been speculated that, in some cases, the bioavailability of a drug is affected by the activity of a multidrug transporter, a membrane-bound P-glycoprotein, which functions as an energy-dependent transport or efflux pump to decrease intracellular accumulation of drug by extruding xenobiotics from the cell. This P-glycoprotein has been identified in normal tissues of secretory endothelium, such as the biliary lining, brush border of the proximal tubule in the kidney and luminal surface of the intestine, and vascular endothelial cells lining the blood brain barrier, placenta and testis.

Therefore, to improve bioavailability of DTSI, the compositions of the invention may have one or more excipients that are P-glycoprotein inhibitors, such as polyoxyethylene 20 sorbitan monooleate (TWEEN 80®), polyoxyl 35 castor oil (CREMOPHOR® EL), polyoxyl 40 castor oil (CREMOPHOR® RH 40), and combinations thereof. Vitamin E TPGS is also a P-glycoprotein inhibitor.

Thus, in one embodiment, the at least one pharmaceutically acceptable excipient is chosen from Vitamin E TPGS, polyethylene glycol, hydroxypropyl methylcellulose, a P-glycoprotein inhibitor, and combinations thereof.

The compositions of the invention may include an additional therapeutic agent. Such additional therapeutic agent may be chosen from a platelet aggregation inhibitor, an HMG-CoA reductase inhibitor, an antihyperlipidemic agent and a cyclooxygenase inhibitor. In one embodiment, the platelet aggregation inhibitor is chosen from tirofiban, dipyridamole, clopidogrel and ticlopidine. The HMG-CoA reductase inhibitor may be chosen from lovastatin, simvastatin, pravastatin, rosuvastatin, mevastatin, atorvastatin, cerivastatin, pitavastatin and fluvastatin. The cyclooxygenase inhibitor may be chosen from rofecoxib, meloxicam, celecoxib, etoricoxib, lumiracoxib, valdecoxib, parecoxib, cimicoxib, diclofenac, sulindac, etodolac, ketoralac, ketoprofen, piroxicam and LAS-34475.

The invention is also directed to a method for the treatment or prophylaxis of a prostaglandin-mediated disease or condition comprising administering to a mammal the compositions described herein. Such prostaglandin-mediated disease or condition may be chosen from pain, fever or inflammation associated with rheumatic fever, influenza or other viral infections, common cold, low back and neck pain, skeletal pain, post-partum pain, dysmenorrhea, headache, migraine, toothache, sprains and strains, myositis, neuralgia, synovitis, arthritis, including rheumatoid arthritis, degenerative joint diseases, gout and ankylosing spondylitis, bursitis, burns including radiation and corrosive chemical injuries, sunburns, pain following surgical and dental procedures, immune and autoimmune diseases; cellular neoplastic transformations or metastatic tumor growth; diabetic retinopathy, tumor angiogenesis; prostanoid-induced smooth muscle contraction associated with dysmenorrhea, premature labor, asthma or eosinophil related disorders; Alzheimer's disease; glaucoma, bone loss; osteoporosis, Paget's disease; peptic ulcers, gastritis, regional enteritis, ulcerative colitis, diverticulitis or other gastrointestinal lesions; GI bleeding; coagulation disorders selected from hypoprothrombinemia, hemophilia and other bleeding problems; kidney disease; thrombosis, myocardial infarction, stroke; and occlusive vascular disease.

In one embodiment, the prostaglandin-mediated disease or condition is occlusive vascular disease. In another embodiment, the invention is directed to a method for reducing plaque in the treatment of atherosclerosis comprising administering to a mammal the above-described composition. In yet another embodiment, the invention is directed to a method for the promotion of bone formation or for cytoprotection comprising administering to a mammal the composition of the invention. In an additional embodiment, the invention is directed to a method for the treatment or prophylaxis of pain, inflammation, atherosclerosis, myocardial infarction, stroke or vascular occlusive disorder comprising administering to a mammal the composition of the invention.

The present invention is also directed to processes of preparation of the described compositions. One such process is a process for preparation of a solid oral pharmaceutical dosage form. The process comprises: a) mixing DTSI, or a pharmaceutically acceptable salt thereof, at least one pharmaceutically acceptable fusible excipient, and, optionally, at least one additional pharmaceutically acceptable excipient; b) subjecting the mixture to injection molding or extrusion; and c) processing the mixture into a dosage form.

In such a process, the mixing step may be carried out at a temperature that is in a range of from about 5 to about 15 degrees C. higher than a melting temperature of the at least one pharmaceutically acceptable fusible excipient. If more than one pharmaceutically acceptable fusible excipients having different melting points are used, the mixing may be carried out at a temperature that is in a range of from about 5 to about 15 degrees C. higher than a melting temperature of a pharmaceutically acceptable fusible excipient with a highest melting point. In such a process, a weight by weight ratio of DTSI, or pharmaceutically acceptable salt thereof, to the at least one pharmaceutically acceptable fusible excipient may be in a range from about 1:15 to about 1:5. However, other ratios described above are also envisioned.

A “fusbile excipient” is an excipient that is solid at room temperature and which can be used to make a solid, single-phase solution with the DTSI. One possible process for preparation of such a single-phase solution is hot-melt extrusion, and another possible process for preparation of such a single-phase solution is injection molding. In one embodiment, the at least one pharmaceutically acceptable fusible excipient is chosen from Vitamin E TPGS, polyethylene glycol, and combinations thereof. In another embodiment, the at least one pharmaceutically acceptable fusible excipient is chosen from Vitamm E TPGS, polyethylene glycol, hydroxypropyl methylcellulose, and combinations thereof. In yet another embodiment, the at least one pharmaceutically acceptable fusible excipient is chosen from Vitamin E TPGS, polyethylene glycol, hydroxypropyl methylcellulose, a P-glycoprotein inhibitor, such as polyoxyethylene 20 sorbitan monooleate (TWEEN 80®), and combinations thereof.

In one embodiment, the above-described subjecting of the mixture to injection molding or extrusion results in a solid, single-phase composition. The at least one additional pharmaceutically acceptable excipient may be choline.

The present invention is also directed to providing improved DTSI solubility formulations in a form of nanoparticle formulations. One way to enhance a drug's bioavailability is to reduce the particle size and distribution range, thereby increasing surface area which speeds up dissolution, and facilitates absorption by the body.

Therefore, in one preferred formulation, DTSI is present as nanoparticles or nanospheres with size ranging between 1-1000 nm prepared using techniques readily available to those skilled in the art. For example, using appropriate excipient combinations, milling processes may be used to break down crystals to obtain particles having a size of 1-1000 nm. After addition of protective excipients, the mixture can be spray dried or freeze dried and formulated as tablets or capsules for oral delivery or as other specific formulations for suitable routes of administration. Alternatively, combination of DTSI with Polyethylene Glycol (PEG) derivatives allows formation of self-assemblies (micelles) incorporating DTSI, thus improving drug solubility and GI absorption. In another approach, supercritical fluid or spray drying technologies are used in combination with appropriate excipients and process manipulation, allowing preparation of DTSI as nano-particles with subsequent incorporation into tablets or capsules for oral delivery. Yet another approach is based on solvent evaporation and coacervation techniques, where DTSI incorporating nano-carriers may be designed using natural polymers (e.g., chitosan, alginate and their derivatives) and artificial polymers (e.g., PLGA, PLA and their derivatives).

Thus, the present invention is also directed to a pharmaceutical composition comprising DTSI, or a pharmaceutically acceptable salt thereof, and at least one pharmaceutically acceptable excipient, wherein DTSI is present as particles in a size range of about 1 nm to about 1000 nm. In one embodiment, the pharmaceutically acceptable salt may be chosen from a salt with a pharmaceutically acceptable primary, secondary, or tertiary amine compound, and a pharmaceutically acceptable quaternary ammonium compound. Such salt may be chosen from a salt with lysine, arginine, betaine, sarcosine, choline, choline phosphate, tromethamine, ethanolamine, diethanolamine, and triethanolamine. The composition may be in a form of a capsule, troche, dispersion, suspension, solution, patch, or a tablet. The at least one pharmaceutically acceptable excipient may be choline.

The above-discussed nanoparticle drug compositions are suitable for oral delivery or other routes of administration, such as by inhalation and by nasal, buccal, sublingual, or rectal delivery.

By way of example, the following procedure may be used to prepare nanoparticle formulations. Nanoparticles may be prepared by the technique reported by Olbrich et al. [Int. J. Pharm. 237, 119-128 (2002)] and by Jenning et al. [J. Microencapsul. 19, 1-10 (2002)]. In brief, DTSI is dissolved in a small quantity of methanol and hydrogenated soya phosphatidylcholine (HSPC) is added. The mixture is warmed to form a clear melt and the methanol is evaporated at 50-55° C. The DTSI-containing HSPC is added to a glyceride lipid chosen from (1) glycerol monostearate (GMS), (2) glycerol distearate (GDS) or (3) tripalmitin (TP) and heated to 5° C. above the melting point of the glyceride lipid to obtain a clear melt. The hot melt is emulsified by stirring for 5 minutes at 5000 rpm into an aqueous phase containing sodium tauroglycocholate, which was preheated to 5° C. above the melting point of the glyceride. The hot emulsion is homogenized in a high pressure homogenizer at 90° C. The nanodispersion thus formed is spray dried at an inlet temperature between 50° C. and 70° C. and outlet temperature of 40° C. with inlet pressure 2.5 kg/cm² and flow rate 3 mL/min.

The nanoparticle preparations include those wherein the drug composition is administered in an effective amount to achieve its intended purpose. More specifically a “therapeutically effective amount” means an amount effective to treat a disease. Determination of the therapeutically effective amount is well within the capability of those skilled in the art, especially in light of the detailed disclosure provided herein.

The exact formulation, route of administration, and dosage is determined by an individual physician in view of the patient's condition. Dosage amount and interval can be adjusted individually to provide levels of the nanoparticle drug composition that are sufficient to maintain therapeutic or prophylactic effect.

The amount of nanoparticle preparation administered is dependent on the subject being treated, on the subject's weight, severity of affliction, the manner of administration, and the judgment of the prescribing physician. Specifically, for administration to a human in curative or prophylactic treatment of a disease, oral dosage of a drug composition is about 10 to about 500 mg daily for an average patient (70 kg). Thus, for a typical adult patient, individual doses contain about 0.1 to 500 mg drug composition, in a suitable pharmaceutically acceptable vehicle or carrier, for administration in single or multiple doses, once or several times per day. Dosages for buccal or sublingual administration typically are in the range of about 0.1 to about 10 mg/kg per single dose, as required. In practice, the physician determines the actual dosing regimen that is most suitable for an individual patient and disease, and the dosage varies with age, weight and response of particular patient. The above dosages are exemplary of average case, but there can be individual instances in which higher or lower dosages are merited, and such are within the scope of this invention.

A nanoparticle drug composition of the present invention can be administered alone or in admixture with a pharmaceutical carrier selected with regard to the intended route of administration and standard pharmaceutical practice. Pharmaceutical nanoparticle preparations for use in accordance with the present invention can be formulated in a conventional manner using one or more pharmaceutical acceptable carriers comprising excipients and auxiliaries that facilitate processing of a drug composition into preparations that can be used pharmaceutically.

The nanoparticle preparations can be manufactured in conventional manner, e.g., by conventional mixing, dissolving, granulating, dragee-making, emulsifying, spray-drying or lyophilizing processes. Proper formulation is dependent upon the route of administration chosen. When a therapeutically effective amount of a drug composition is administrated orally, the formulation typically is in the form of a tablet, capsule, powder solution, suspension or elixir.

When administrated in tablet form the nanoparticle composition additionally can contain a solid carrier, such as gelatin or an adjuvant. The tablet, capsule and powder contain about 5% to about 95%, preferably about 25% to about 90% of a drug composition of the present invention.

When administered in a liquid form, a liquid carrier, such as water, petrolatum or oils of animal or plant origin can be added. The liquid form of nanoparticle preparation can further contain physiological saline solution, dextrose or other saccharide solutions or glycols. When administered in liquid form, the nanoparticle preparation contains about 0.5% to about 90%, by weight, of drug composition and preferably about 1% to about 50%, by weight, of drug composition.

The nanoparticle drug composition can be readily combined with pharmaceutically acceptable carriers well-known in the art. Such carriers enable the nanoparticle drug composition to be formulated as tablets, pills, dragee, capsules, liquids, gels, syrups, slurries, suspensions and the like, for oral ingestion by a patient to be treated.

Pharmaceutical nanoparticle preparations for oral use can be obtained by adding to the drug composition a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The nanoparticle drug composition also can be formulated as a rectal composition, such as suppositories or retention enemas, e.g. containing conventional suppositories bases.

In particular, the nanoparticle drug composition can be administrated orally, buccally or sublingually in the form of tablets containing excipients such as starch or lactose or in capsules either alone or in admixture with excipients or in form of elixirs or suspensions containing flavoring or coloring agents.

It has also been discovered that micronized DTSI has particularly good aqueous solubility of 46.6 mg/mL when in a solution of 5% Vitamin E TPGS and 1% choline. Even higher DTSI solubility of 58.5 mg/mL was observed in a solution of 5% Vitamin E TPGS and 5% diethanolamine. Experiments also showed micronized DTSI solubility of: 15.6 mg/mL in 5% Vitamin E TPGS 5% and 0.5% diethanolamine; and 20.4 mg/mL in 5% Vitamin E TPGS and 1% diethanolamine. In other experiments, micronized DTSI showed solubility of: 0.10 mg/mL in 5% Vitamin E TPGS; 8.5 mg/mL in 5% Vitamin E TPGS 5% and 1% tromethamine; 19.2 mg/mL in 5% Vitamin E TPGS and 1% ethanolamine; 20.4 mg/mL in 5% Vitamin E TPGS and 1% diethanolamine; and 9.2 mg/mL in 5% Vitamin E TPGS and 1% triethanolamine. In another set of experiments, micronized DTSI showed solubility of: 0.17 mg/mL in 5% Cremophor® RH40; 8.7 mg/mL in 5% Cremophor® RH40 5% and 1% tromethamine; 8.9 mg/mL in 5% Cremophor® RH40 and 1% ethanolamine; 22.7 mg/mL in 5% Cremophor® RH40 and 1% diethanolamine; and 8.3 mg/mL in 5% Cremophor® RH40 and 1% triethanolamine.

Thus, in one embodiment, the invention is directed to a pharmaceutical composition comprising a pharmaceutically acceptable salt of DTSI and at least one pharmaceutically acceptable excipient, wherein the pharmaceutically acceptable salt is chosen from a salt with a pharmaceutically acceptable primary, secondary, or tertiary amine compound, and a pharmaceutically acceptable quaternary ammonium compound. The pharmaceutically acceptable salt may by chosen from a salt with lysine, arginine, NN-dimethyl glycine, betaine, sarcosine, choline, choline phosphate, tromethamine, ethanolamine, diethanolamine, and triethanolamine. The composition may be in a form of a as tablets, pills, dragee, capsules, liquids, gels, syrups, slurries, dispersion, troche, patch, or suspensions. The at least one pharmaceutically acceptable excipient may be choline.

### In Vitro and In Vivo Utility of DTSI

DTSI and related compounds were assayed for binding on prostanoid hEP3 receptors according to the method of Abramovitz et al [Bioch. Biophys. Acta, 1473, 285-293 (2000)]. DTSI exhibited an IC₅₀ 4.6 nM.

DTSI and related compounds were assayed for their effect on platelet aggregation in vitro. In experiments with human platelets, whole blood was extracted from overnight-fasted human donors. Each experiment was performed with blood from a single individual. In experiments with rodent platelets, whole blood was gathered from the heart of female mice or male rats under isofluran (Abbott) anaesthesia. Blood was pooled from two or ten individual rodents for each experiment in the case of rat and mouse experiments, respectively. In all cases, blood was collected into 3.8% sodium citrate tubes (Greiner Bio-one). Platelet-rich plasma (PRP) was obtained by centrifugation at 100×g for 15 min at 25° C. for humans, at 150×g for rats, or at 80×g for 10 min for mice. Platelet-poor plasma was obtained by centrifugation of the remaining blood at 2,400×g for 10 min at 25° C. After counting in an Autocounter (Model 920 EO, Swelab) platelets were diluted when necessary to the desired stock concentrations (200,000-300,000 platelets/μl) using 0.9% NaCl isotonic solution (Braun).

Platelet aggregation was determined by light absorbance using a platelet aggregometer with constant magnetic stirring (Model 490, Chronolog Cop., Havertown, Pa., USA), using a volume of 500 μl per cuvette. During the performance of the experiments, platelet solution was continually agitated by mild horizontal shaking. Collagen (Sigma) and sulprostone (Cayman Chemicals) were used as accelerants of platelet aggregation. Compounds used for this assay were dissolved and stored in a 100% DMSO solution. After dilution, the final DMSO concentration in the assay was lower than 0.1% v/v. It was determined that this concentration of DMSO did not inhibit platelet aggregation in the assay. Acceleration agents and EP₃ test compounds were diluted in isotonic solution at the desired concentration. Sigmoidal non-lineal regression was used to calculate the concentration of test compound required to inhibit platelet aggregation by 50% (IC₅₀). IC₅₀ values were calculated using GraphPad Prism 3.02 for Windows (GraphPad Software, San Diego, Calif. USA). The data are shown in Table 1, where Sulprostone (100 nM) and collagen (0.125 ug.mL) were used for human and Sulprostone (100 nM) and collagen (2.0 ug.mL) were used for rat assays.

TABLE 1
IC₅₀ values of DTSI in the platelet aggregation assay.

| Species | Agonist | Serum % | IC₅₀ (nM) |
|---|---|---|---|
| Human | Sulprostone | 100 | 218 |
| Rat | Sulprostone | 20 | 83 |

DTSI was also assayed for its effects on platelet aggregation in vivo. An in vivo test of platelet activation is the induction of pulmonary thromboembolism by arachidonic acid, a precursor of prostaglandin formation. Inhibitors of prostaglandin synthesis, e.g., a COX-1 inhibitor such as asprin, are protective in the assay. For the pulmonary thromboembolism assay, conscious female C57BL/6 mice were dosed orally with the test compounds and 30 min later thromboembolism was induced by injection of arachidonic acid into a tail vein at a dose of 30 mg per kg body weight. Survival was evaluated one hour after the challenge with arachidonic acid, as mice that survive for that length of time usually recovered fully. The arachidonic acid injection was given via a lateral tail vein in a mouse that had been warmed briefly under a heat lamp (dilation of the tail veins using heat facilitates placing the injection). An insulin syringe, 0. 5 mL (from Becton Dickinson) was used for dosing. The dose volume given of both the test compound and the arachidonic acid was adjusted to the weight of the mouse (the dose volume p.o. for test compounds and i.v. for arachidonic acid solution was 10 μL and 5 μL per gram body weight, respectively). The survival rate for mice treated with arachidonic acid only was 1 per 10 mice evaluated or 10%. Survival rate for mice treated with the DTSI (100 mg/kg, orally) and then arachidonic acid was 68% (15 mice surviving out of 22 mice evaluated).

Thus, DTSI is a potent, selective EP3 receptor antagonist.

Method of Preparation of DTSI.

### EXAMPLE 1

### Preparation of methyl 3-(5-fluoro-3-methylindol-7-yl)acrylate via Heck Coupling with Isolation of 7-haloindole Intermediate

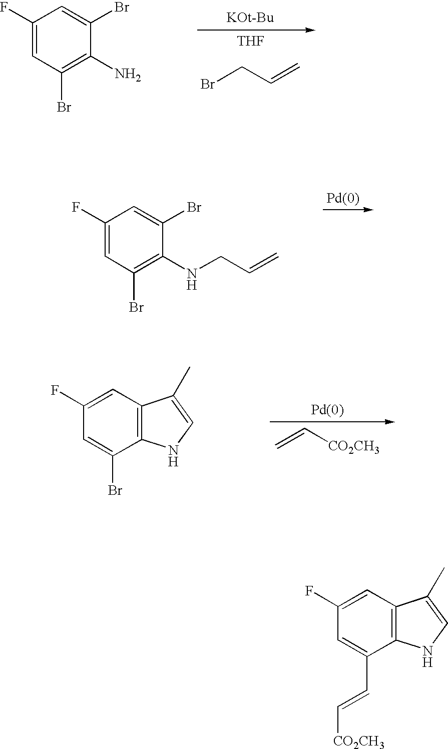

Step 1. N-Allyl-2,6-dibromo-4-fluoroaniline. 2,6-Dibromo-4-fluoroaniline (100 g, 0.372 mole) was charged into a 3-neck 3 L flask fitted with mechanical stirrer and dissolved in anhydrous tetrahydrofuran (500 mL). To this solution was charged a solution of KOtBu (1.0 M in THF, 465 mL, 0.465 mole). Allyl bromide (37 mL, 0.427 mole) was added via an addition funnel over 20 min. The mixture was stirred at ambient temperature for 14 h. The reaction mixture was diluted with MTBE (1.0 L), and water (1.0 L). The upper organic layer was separated, washed with water (2×600 mL) and brine, then dried over sodium sulfate. After filtration the solvent was removed to obtain 118 g of a brown oil. The oil was chromatographed over silica gel (500 g) and eluted with hexanes. The fractions containing the desired product were pooled and concentrated to yield 112 g (97% yield) of the desired product as a yellow oil: ¹H NMR (CDCl3) δ 3.75 (br s, 1H), 3.79 (d, 2H, J=6.4 Hz), 5.13 (dd, 1H, J=9.6, 0.8 Hz), 5.26 (dt, 1H, J=16.8, 0.8 Hz), 5.97 (m, 1H), 7.27 (d, 2H, J=7.6 Hz).

Step 2. 7-Bromo-5-fluoro-3-methylindole. To a solution of N-Allyl-2,6-dibromo-4-fluoroaniline (20 g, 65 mmol) in 100 mL acetonitrile was added palladium(II) acetate (150 mg, 0.7 mmol), tri-O-tolylphosphine (600 mg, 2 mmol) and triethylamine (26.3 g, 260 mmol), and the resulting solution was heated at reflux for 2.5 h. The reaction was cooled to room temperature and filtered through a celite mat. The celite was rinsed with 25 mL acetonitrile, and the combined solutions were concentrated in vacuo to provide 22.5 g of crude product. The product was purified via silica gel column chromoatography to afford 11.3 g (77% yield) of the title compound: ¹H NMR (400 MHz, CDCl₃) δ 2.27 (d, 3H, J=1.2 Hz), 7.06 (br s, 1H), 7.14 (dd, 1H, J=8.8, 2.4 Hz), 7.18 (dd, 1H, J=8.8, 2.4 Hz), 8.01 (br, 1H).

Step 3. Methyl 3-(5-fluoro-3-methylindol-7-yl)acrylate. To a solution of 7-bromo-5-fluoro-3-methylindole (1.145 kg, 5.02 moles) in 6.9 L acetonitrile was added methyl acrylate (904 mL, 10.04 moles), palladium(II) acetate (56.3 g, 250 mmol), tri-O-tolylphosphine (229 g, 750 mmol), and triethylamine (4.2 L, 30 moles), and the solution was heated at reflux for 16 h. After cooling to room temperature, the solution was diluted with 5.5 L water and 4.5 L MTBE. The organic phase was separated and washed with water and brine, dried over anhydrous sodium sulfate, and filtered through a celite mat. Concentration in vacuo afforded the crude product as an orange solid (1.6 kg). The solid was slurried with 3 L of hexanes for 1.5 h, then collected via filtration, rinsed with hexanes and air dried, to afford the pure title product in quantitative yield. The material could be further purified via silica gel column chromatography: ¹H NMR (400 MHz, CDCl₃) δ 2.29 (d, 3H, J=1.2 Hz), 3.84 (s, 3H), 6.49 (d, 1H, J=16 Hz), 7.07 (br s, 1H), 7.15 (dd, 1H, J=10, 2.4 Hz), 7.27 (dd, 1H, J=9.2, 2.4 Hz), 7.95 (d, 1H, J=16 Hz), 8.35 (br s, 1H).

### Example 2

### Preparation of methyl 3-(5-fluoro-3-methylindol-7-yl)acrylate via Heck Coupling without Isolation of 7-haloindole Intermediate

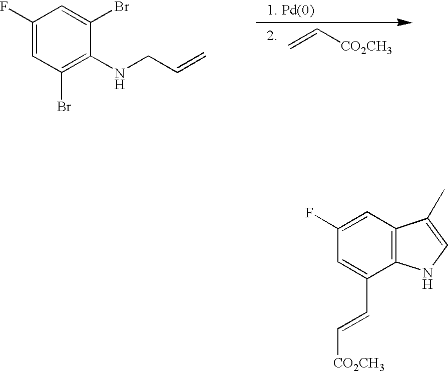

To a solution of N-allyl-2,6-dibromo-4-fluoroaniline (23.0 g, 74.4 mmol), prepared as in Step 1 of Example 1, in anhydrous acetonitrile (115 mL) in a 3-neck 250 mL flask fitted with a condenser, temperature probe, heating mantle, and nitrogen bubbler was added palladium(II) acetate (167 mg, 0.744 mmol), tri-O-tolylphosphine (906 mg, 3.0 mmol), and triethylamine (15.6 mL, 110 mmol). The dark solution was refluxed under nitrogen. After 2 h, TLC analysis indicated that the starting material was consumed. After two additional h the reaction mixture was cooled to ˜40° C., and the solution was charged with palladium(II) acetate (167 mg), tri-O-tolylphosphine (906 mg), triethylamine (15.6 mL), and methyl acrylate (13.4 mL, 149 mmol), and reflux was resumed. After cooling to room temperature the reaction mixture was diluted with MTBE (200 mL) and water (200 mL), and the mixture was stirred for 10 min. The dark upper organic layer was separated and washed with water (3×100 mL), brine (100 mL), and dried over sodium sulfate. After filtration the solvent was removed to obtain a tan solid. The material was dried at 50° C. for 2 h, providing 19.3 g (111%) of crude product. The crude material was suspended in a mixture of MTBE (60 mL) and hexanes (100 mL), and the mixture was refluxed for 2 h. After cooling to room temperature a gray-colored solid was collected by filtration, washed well with hexane (200 mL), and dried under vacuum at 45-50° C. for 60 hrs, providing 7.2 g of the desired product: ¹H NMR (400 MHz, CDCl₃) δ 2.29 (d, 3H, J=1.2 Hz), 3.84 (s, 3H), 6.49 (d, 1H, J=16 Hz), 7.07 (br s, 1H), 7.15 (dd, 1H, J=10, 2.4 Hz), 7.27 (dd, 1H, J=9.2, 2.4 Hz), 7.95 (d, 1H, J=16 Hz), 8.35 (br s, 1H).

### Example 3

### Preparation of 3-(5-fluoro-3-methylindol-7-yl)acrylic Acid via Heck Coupling without Isolation of 7-haloindole Intermediate

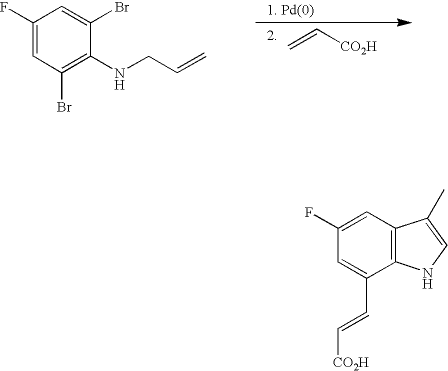

To a solution of N-allyl-2,6-dibromo-4-fluoroaniline (2.09 g, 6.76 mmol), prepared as in Step 1 of Example 1, in anhydrous acetonitrile (15 mL) was added palladium(II) acetate (31.4 mg, 0.137 mmol), tri-O-tolylphosphine (120 mg, 0.383 mmol), and triethylamine (3.8 mL, 27.3 mmol). The reaction was heated at reflux for 3 h, at which point TLC indicated consumption of starting material. The reaction was cooled to room temperature, then acrylic acid (0.56 mL, 8.08 mmol) was added via syringe and refluxing was resumed. After 3.5 h at reflux, TLC indicated reaction completion. The solution was cooled to room temperature, diluted with 21 mL water, then approximately 10 mL of the solvent was evaporated in vacuo. The solution was diluted with additional water and washed with MTBE (2×10 mL). The separated aqueous solution was acidified to pH 2-3 with 1 M HCl, which induced precipitation of the product as a yellow solid. The product was collected via suction filtration, washed with water, then vacuum dried overnight at 47° C., providing the title compound as a bright yellow solid (1.33 g, 90% yield): ¹H NMR (400 MHz, DMSO-d₆) δ 2.23 (d, 3H, J=0.8 Hz), 6.67 (d, 1H, J=16 Hz), 7.24 (br s, 1H), 7.34 (dd, 1H, J=9.2, 2.4 Hz), 7.41 (dd, 1H, J=10.4, 2.4 Hz), 8.06 (dd, 1H, J=16, 1.2 Hz), 11.35 (s, 1H).

### Example 4

### Preparation of methyl 3-(1-(2,4-dichloro)benzyl-5-fluoro-3-methylindol-7-yl)acrylate with Isolation of 7-haloindole Intermediate

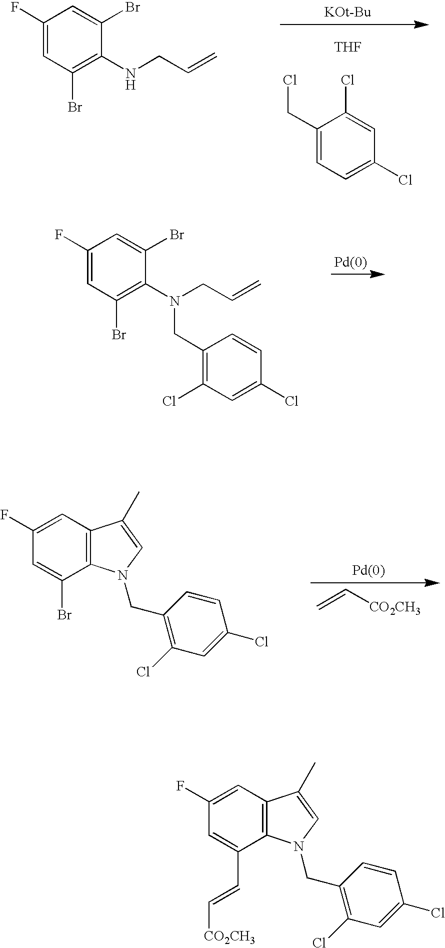

Step 1. N-Allyl-N-(2,4-dichloro)benzyl-2,6-dibromo-4-fluoroaniline. N-Allyl-2,6-dibromo-4-fluoroaniline (8.0 g, 25.9 mmol), prepared as described in Step 1 of Example 1, was dissolved in 80 mL THF. A solution of potassium t-butoxide in THF (1 M, 51.7 mmol) was added via syringe, and stirring was continued for 1 h. 2,4-Dichlorobenzyl chloride (6.1 g, 31.2 mmol) was added via syringe, and the reaction was stirred at room temperature for 24 h. The reaction mixture was diluted with ethyl acetate and washed sequentially with water and brine, dried over sodium sulfate, and concentrated to afford 10.7 g (90% yield) of the desired product as a brown semi-solid. The product could be further purified via recrystallization from methanol or acetonitrile: ¹H NMR (400 MHz, CDCl₃) δ 3.77 (d, 2H, J=5.6Hz), 4.39 (s, 2H), 5.05 (dd, 1H, J=9.6, 0.8 Hz), 5.15 (dt, 1H, J=16.8, 0.8 Hz), 5.95 (m, 1H), 7.1-7.5 (m, 5H).

Step 2. 7-Bromo-1-(2,4-dichloro)benzyl-5-fluoro-3-methylindole. To a solution of N-Allyl-N-(2,4-dichloro)benzyl-2,6-dibromo-4-fluoroaniline (10.0 g, 21 mmol) in 50 mL acetonitrile was added palladium(II) acetate (470 mg, 2 mmol), tri-O-tolylphosphine (1.92 g, 6 mmol) and triethylamine (3.19 g, 32 mmol), and the resulting solution was heated at reflux for 17 h. The reaction was cooled to room temperature and filtered through a celite mat. The solution was concentrated in vacuo and the residue was partitioned between EtOAc and water. The organic phase was washed with water and brine, dried over anhydrous sodium sulfate, filtered and concentrated to provide 7.7 g of crude product. The product was purified via silica gel column chromatography with hexanes to afford 1.7 g (23% yield) of the desired product. ¹H NMR (400 MHz, CDCl₃) δ 2.17 (s, 3H), 5.69 (s, 2H), 6.22 (d, 1H, J=8.4 Hz), 6.89 (s, 1H), 7.05 (dd, 1H, J=8.4, 2.0 Hz), 7.12 (dd, 1H, J=8.8, 2.4 Hz), 7.19 (dd, 1H, J=8.8, 2.4 Hz), 7.41 (d, 1H, J=2.0 Hz).

Step 3. Methyl 3-(1-(2,4-dichloro)benzyl-5-fluoro-3-methylindol-7-yl)acrylate. To a solution of 7-bromo-1-(2,4-dichloro)benzyl-5-fluoro-3-methylindole (4.1 g, 11 mmol) in 40 mL THF was added palladium(II) acetate (0.47 g, 2 mmol), tri-O-tolyl)phosphine (1.92 g, 6 mmol), and triethylamine (3.19 g, 32 mmol), and the reaction was heated at reflux for 17 h. The mixture was cooled to room temperature, filtered through a celite mat, and concentrated under reduced pressure. The residue was partitioned between EtOAc and water, and the separated organic phase was washed sequentially with water and brine. The solution was dried over sodium sulfate, filtered and concentrated to afford the crude product (7.7 g). Purification via silica gel chromatography (hexanes) afforded 17 g (23% yield) of the desired title compound: ¹H NMR (400 MHz, CDCl₃) δ 2.30 (d, 3H, J=0.8 Hz), 3.74 (s, 3H), 5.43 (s, 2H), 6.19 (d, 1H, J=15.4 Hz), 6.32 (d, 1H, J=8.8 Hz), 6.90 (br s, 1H), 7.02 (dd, 1H, J=10.0, 2.4 Hz), 7.06 (dd, 1H, J=8.6, 2.0 Hz), 7.27 (dd, 1H, J=8.6, 2.4 Hz), 7.47 (d, 1H, J=2.0 Hz), 7.75 (d, 1H, J=15.4 Hz).

Further elaboration of the products of the processes of cyclization and acrylate addition:

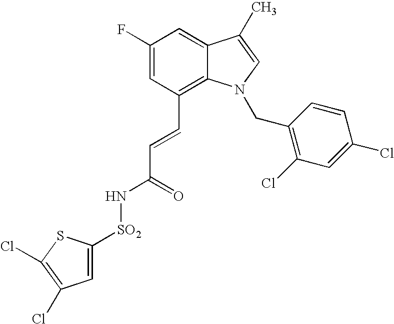

4,5-Dichloro-thiophene-2-sulfonic acid [(E)-3-[1-(2,4-dichlorophenylmethyl)-5-fluoro-3-methyl-1H-indol-7-yl]-acryloyl]amide (DTSI)

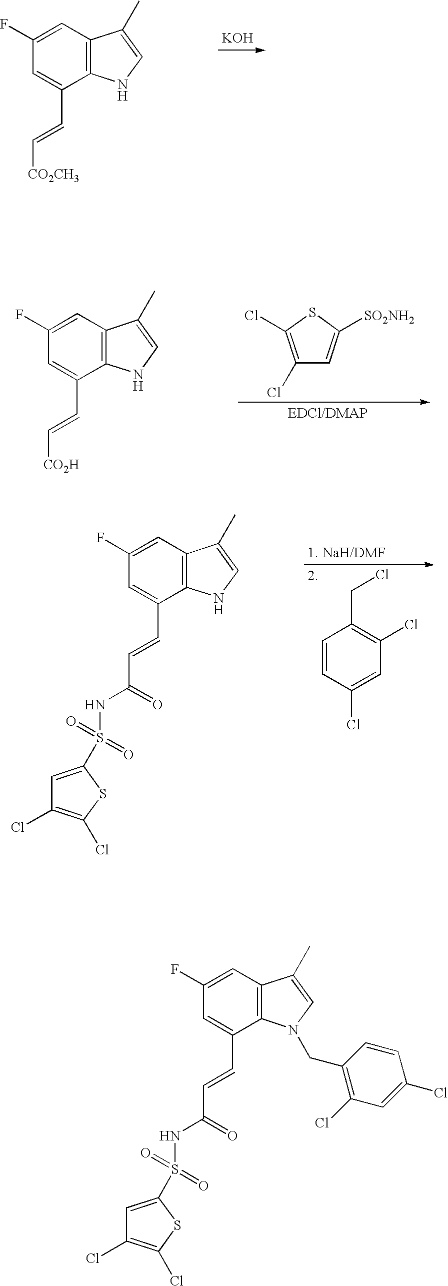

Synthesis of (E)-3-(5-Fluoro-3-methyl-1H-indol-7-yl)-acrylic acid. To a stirred solution of methyl 3-(5-fluoro-3-methylindol-7-yl)acrylate (1.75 kg, 7.51 mole), prepared as described in Example 1, in 23.4 L THF/MeOH (1:1) at room temperature was added 2 M aqueous sodium hydroxide (16.35 L, 32.7 moles). Stirring was continued for 15 h, then the reaction mixture was concentrated in vacuo to remove the volatile organic solvents. The solution was diluted with 20 L water, then extracted with dichloromethane (3×10 L). The aqueous layer was acidified to a pH of 2-3 with 2 M HCl, which induced precipitation of the product. The product was collected via vacuum filtration, washed with water (2×2 L), and vacuum dried at 60° C. to afford 1.036 kg (91% yield) of the desired title compound: ¹H NMR (400 MHz, DMSO-d₆) δ 2.23 (d, 3H, J=0.8 Hz), 6.67 (d, 1H, J=16 Hz), 7.24 (br s, 1H), 7.34 (dd, 1H, J=9.2, 2.4 Hz), 7.41 (dd, 1H, J=10.4, 2.4 Hz), 8.06 (dd, 1H, J=16, 1.2 Hz), 11.35 (s, 1H).

Synthesis of 4,5-dichlorothiophene-2-sulfonic acid [(E)-3-(5-fluoro-3-methyl-1H-indol-7-yl)-acryloyl]-amide. A mixture of (E)-3-(5-fluoro-3-methyl-1H-indol-7-yl)-acrylic acid (772 g, 3.53 mole), 4,5-dichloro-2-thiophenesulfonamide (900 g, 3.88 mole), 4-(dimethylamino)pyridine (861 g, 7.06 mole) and EDCI (1.348 kg, 7.06 mole) in dichloromethane (25.5 L) was stirred at ambient temperature for 14 h. The solution was diluted with 2 M aqueous HCl (16 L), and stirred for 1.5 h, which induced precipitation of the product. The product was collected via vacuum filtration and washed sequentially with water (2×2 L), dichloromethane (2×2 L), and hexanes (2 L) to provide 1.044 kg (71% yield) of the desired title compound. ¹H NMR (400 MHz, DMSO-d₆) δ 2.23 (s, 3H), 6.71 (d, 1H, J=15.6 Hz), 7.22 (dd, 1H, J=10.0, 2.6 Hz), 7.27 (br s, 1H), 7.39 (dd, 1H, J=9.6, 2.6 Hz), 7.95 (s, 1H), 8.15 (dd, 1H, J=15.6, 1.2 Hz), 11.35 (s, 1H).

Synthesis of 4,5-dichloro-thiophene-2-sulfonic acid [(E)-3-[1-(2,4-dichlorophenylmethyl)-5-fluoro-3-methyl-1H-indol-7-yl]-acryloyl]amide (DTSI). To a solution of 4,5-dichlorothiophene-2-sulfonic acid [(E)-3-(5-fluoro-3-methyl-1 H-indol-7-yl)-acryloyl]-amide (1.025 kg, 2.37 mole) in DMF (5.1 L) at 0° C. was added NaH (60% in oil, 353 g, 8.8 mole) portionwise and the reaction mixture was allowed to stir for 30 min. 2,4-Dichlorobenzyl chloride (924 g, 1.41 mole) was added at such a rate to maintain the temperature near 0° C. After stirring about 45 min, the reaction mixture was carefully quenched with water (15 L), then diluted with 2 M HCl (9 L) and dichloromethane (10 L), which led to precipitation of the desired title product. The precipitated product was collected via vacuum filtration and the filter cake was washed sequentially with water (2×2 L), and cold EtOH (2×1 L). The product was vacuum dried at 60° C. to afford 1.305 kg (93% yield) of desired product, as a solvate with DMF. The product was recrystallized from absolute EtOH to afford the pure product: ¹H-NMR (400 MHz, DMSO-d₆) δ 2.26 (s, 3H), 5.53 (s, 2H), 6.12 (d, 1H, J=8.4 Hz), 6.21 (d, 1H, J=15.4 Hz), 7.04 (dd, 1H, J=10.0, 2.4 Hz), 7.22 (dd, 1H, J=8.4, 2.0 Hz), 7.37 (s, 1H), 7.38 (d, 1H, J=2.0 Hz), 7.46 (dd, 1H, J=9.2, 2.4 Hz), 7.74 (d, 1H, J=15.4 Hz), 7.90 (s, 1H).

DTSI via an Alternative Route

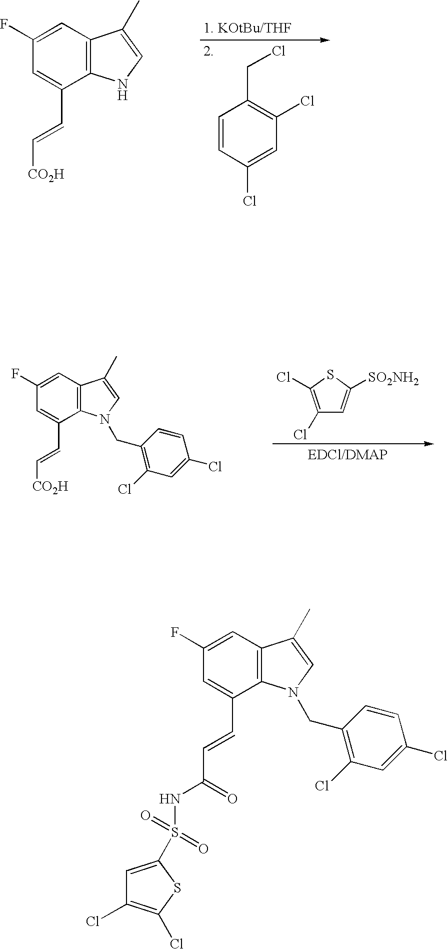

Synthesis of (E)-3-[1-(2,4-Dichlorobenzyl)-5-fluoro-3-methyl-1H-indol-7-yl]-acrylic acid. To a solution of 3-(5-fluoro-3-methylindol-7-yl)acrylic acid, prepared as in Example 3 (20 g, 92 mmol) in 200 mL THF was added potassium t-butoxide (24.4 g, 206 mmol) in portions over approximately 10 min, while keeping the internal temperature below 18° C. with an ice-water bath. 2,4-Dichlorobenzyl chloride (21.7 g, 110 mmol) was added over a period of 5 min, after which the cooling bath was removed. The reaction mixture was stirred for 24 h, then quenched with 200 mL water, followed by dilution with 200 mL MTBE and 200 mL heptanes. After stirring for 10 min, the layers were separated, and the aqueous layer was filtered through a celite pad. The pad was rinsed with 50 mL water, and the aqueous filtrate was acidified to pH of 1-2 with 2 M HCl. The suspension was diluted with 200 mL MTBE and 100 mL heptanes, stirred for 5 min, then the solids were collected on a fritted glass funnel and rinsed with heptanes. The solids were dried under reduced pressure overnight at 58° C. to afford 24.4 g (70% yield) of the title compound: ¹H-NMR (400 MHz, DMSO-d₆) δ 2.26 (s, 3H), 5.55 (s, 2H), 6.21 (d, 1H, J=8.4 Hz), 6.24 (d, 1H, J=15.6 Hz), 7.22 (dd, 1H, J=10.4, 2.4 Hz), 7.28 (dd, 1H, J=8.6, 2.0 Hz), 7.34 (s, 1H), 7.43 (dd, 1H, J=8.6, 2.4Hz), 7.66 (d, 1H, J=15.6 Hz), 7.67 (d, 1H, J=2.4 Hz), 12.29 (s, 1H).

Synthesis of 4,5-Dichloro-thiophene-2-sulfonic acid [(E)-3-[1-(2,4-dichlorophenylmethyl)-5-fluoro-3-methyl-1H-indol-7-yl]-acryloyl]amide (DTSI). To a solution of (E)-3-[1-(2,4-dichloro-benzyl)-5-fluoro-3-methyl-1H-indol-7-yl]-acrylic acid (10.0 g, 26.4 mmol) in dichloromethane (100 mL) was added EDCI (7.9 g, 41.2 mmol), HOBt hydrate (0.71 g, 5.3 mmol), and diisopropylethylamine (10.6 g, 81.8 mmol), and the mixture was stirred for 20 min. To the reaction was added 2,4-dichlorothiophene-2-sulfonamide (6.43 g, 27.2 mmol), and the mixture was stirred at room temperature for 15 min, then at reflux for 16 h. The reaction was cooled to room temperature then diluted with 25 mL water followed by 25 mL 2 M HCl. The mixture was stirred for 5 min, then the phases were split. The organic phase was diluted with 25 mL of 2 M HCl and stirred, which induced precipitation of the product. The temperature was reduced to 0° C., and stirring was continued for 1 h. The product was collected via vacuum filtration, washed with water (3×25 mL) and heptanes (2×25 mL), then vacuum dried at 60° C. to afford 9.3 g (60% yield) of the title compound. The product could be further purified via recrystallization from ethanol: ¹H-NMR (400 MHz, DMSO-d₆) δ 2.26 (s, 3H), 5.53 (s, 2H), 6.12 (d, 1H, J=8.4 Hz), 6.21 (d, 1H, J=15.4 Hz), 7.04 (dd, 1H, J=10.0, 2.4 Hz), 7.22 (dd, 1H, J=8.4, 2.0 Hz), 7.37 (s, 1H), 7.38 (d, 1H, J=2.0 Hz), 7.46 (dd, 1H, J=9.2, 2.4 Hz), 7.74 (d, 1H, J=15.4 Hz), 7.90 (s, 1H).

### Formulations.

DTSI exhibits poor aqueous solubility. Therefore, a number of soluble, suspension and solid dosage forms were evaluated. Several of these were subsequently used for obtaining rat pharmacokinetic (PK) parameters following gavage and capsule dosing. In the examples below, DTSI is synonymous with the term “active pharmaceutical ingredient” or “API.”

1. Liquid Formulations: Homogenous

The solubility of DTSI in aqueous PEG, cyclodextrins, glycerides and other solvent systems was evaluated. Excess amount of the DTSI was added to the aqueous compatible/complexation media, the suspension formed was sonicated for 60 minutes (and also heated up to 75° C. e.g. for cyclodextrins). The suspensions at room temperature were filtered and the amount of dissolved drug was determined by HPLC.

2. Liquid Formulations: Suspensions

Formulations containing Ora-Plus, Ora-Plus and cyclodextrin, SDS, Labrasol® and methyl cellulose were utilized to prepare suspension formulations. These preparations were homogenized with Ultra Turex T25 homogenizer to produce small particles and to provide a consistent and reproducible formulation. Aliquots of these samples were mixed with DMSO to fully solubilize the API for analysis by HPLC in order to determine the strength of these formulations.

3. Solid Formulations: Dry Mix

Mixing of the dry ingredients, including solid API, was carried out using a mortar and pestle. Dry ingredient examples as was done for liquid and suspension? After through mixing, samples were taken in DMSO similar to those described for suspensions above. The dissolved DTSI was filtered and measured by HPLC.

4. Solid Formulations: Hot-Melt

The exciplents, which are solid at room temperature, were mixed with the API (solid) and the mixture was heated to melt and dissolve the API. The homogenous melt, upon cooling to room temperature provided solid mass, contained solid API. These formulations are referred to here as Hot-Melt formulations.

### Pharmacokinetic Testing of Formulations.

A number of diverse types of formulations were used for obtaining pharmacokinetic parameters following oral dosing as capsules to rats. Subsequently, more promising formulations were dosed orally in dogs for obtaining pharmacokinetic parameters.

1. Liquid Formulations (Dosed as Capsules)

Some of the solution formulations were dosed orally as capsules to rats for pharmacokinetic studies and the summary of the pharmacokinetic parameters (Tₘₐₓ, Cₘₐₓ and AUC_{0-6 hr}) for selected liquid formulations is shown below in Table 2.

TABLE 2
Rat Pharmacokinetic Parameters^{#} for Liquid Formulations Dosed as Capsules

| | | | | | Conc. in |
|---|---|---|---|---|---|
| Dose | Tmax | Rel. | Rel. | | mg/mL for |
| (mg/kg) | (hr) | Cmax | AUC | Fomulation | Capsule |
| 30 | 1.3 | 143 | 128 | Labrosol (100%) in Gelatin Capsules | 30 |
| 30 | 2.0 | 27 | 43 | Lauroyl Glycol (100%) in Gelatin Capsules | 37.5 |
| 30 | 1.7 | 52 | 22 | SEDDS in Gelatin Capsules | 37.5 |
| 30 | 1.5 | 71 | 54 | PEG400/Gelucrie (1:6) in Gelatin Capsules | 37.5 |
| 30 | 1.3 | 119 | 79 | PEG400/Tween80 (4:1) in Gelatin Capsules | 37.5 |
| 30 | 1.5 | 263 | 133 | PEG3350/Tween80 (3:1) in Gelatin Capsules | 37.5 |
| 10 | 6.0 | 113 | 194 | PEG3350/Tween80 (3:1) in Gelatin Capsules | 37.5 |
| 10 | 1.0 | 629 | 260 | PEG3350/Tween80 (3:1) in Gelatin Capsules | 37.5 |
| 30 | 1.7 | 119 | 97 | PEG3350/Tween80 (3:1) in Gelatin Capsules | 37.5 |
| 10 | 1.7 | 100 | 100 | Micronised powder (DTSI) (100%) in Gelatin | NA |
| | | | | Capsules | |
| 30 | 2.0 | 100 | 100 | Micronised powder (DTSI) (100%) in Gelatin | NA |
| | | | | Capsules | |

^{#}Relative Ratio of Cₘₐₓ and AUC shown is compared with same doses (10 and 30 mg/kg, respectively) of the micronized powder

2. Solid Dry-Mix Formulations (Capsules)

The following materials were used to prepare a number of dry-mix formulations and some were evaluated in rat pharmacokinetics when dosing in capsules: Cyclodextrin lyophilized powder, CABOSIL, AVICEL®, SDS, Dibasic calcium phosphate, and Lactose Monohydrate.

A summary of rat oral pharmacokinetic data, highlighting Cₘₐₓ, and AUC (relative to the micronized powder) is shown in Table 3, and is compared to micronized powder itself, as dosed in a capsule. All of the dry mix formulations showed inferior pharmacokinetic profile vs. micronized powder.

TABLE 3
Rat oral Pharmacokinetic Parameters, formulations dosed orally
as capsules (n = 3/formulation).

| Dose | Tmax | Rel | Rel. | |
|---|---|---|---|---|
| (mg/kg) | (hr) | Cmax# | AUClast# | Fomulation |
| 10 | 1.3 | 55 | 26 | Avicel:Miconized powder |
| | | | | DTSI (1:1) w/ 0.5% |
| | | | | SDS {1-capsule] |
| 30 | 2.0 | 28 | 28 | Avicel:Miconized powder |
| | | | | DTSI (1:1) w/ 0.5% |
| | | | | SDS {3-capsule] |
| 10 | 2.0 | 128 | 60 | Avicel:Miconized powder |
| | | | | DTSI (1:1) w/ 0.5% |
| | | | | SDS {1-capsule/anstehtized] |
| 30 | 3.3 | 25 | 38 | Avicel:DTSI (1:1) in Gelatin |
| | | | | Capsules |
| 11.2 | 1.5 | 81 | 33 | Micronised powder |
| | | | | (DTSI) (99.5%) + 0.5% |
| | | | | SDS |
| 11.2 | 1.5 | 79 | 41 | Micronised powder |
| | | | | (DTSI) (99%) + 1.0% |
| | | | | SDS |
| 10 | 1.7 | 100 | 100 | Micronised powder (DTSI) (100%) |
| | | | | in Gelatin Capsule |
| 30 | 2.0 | 100 | 100 | Micronised powder (DTSI) (100%) |
| | | | | in Gelatin Capsule |

#Relative Ratio of Cmax and AUC shown is compared with same doses (10 and 30 mg/kg, respectively) of the micronized powder.

3. Hot-Melt Formulations

Even though some PEG3350 and LABRASOL® derived formulations showed superior pharmacokinetic profile (higher Cₘₐₓ and AUC) when compared to the micronized powder, based on the solubility profile in these formulations with a single excipient, one would need multiple capsules for human dosing (100 mg—projected human dose). Formulations that retain or even provide further improved pharmacokinetic profile while significantly improving the APT load would be more advantageous. Therefore, various combinations and proportions of the following excipients were evaluated.

1.1.1 Vitamin E TPGS-(d-α-tocopheryl Polyethelene Glycol 1000 Succinate) from Eastman (lot#3005000),

1.1.2. Labrasol® (Caprylocaproyl Polyoxglycerides) from Gattefosse (lot#34098),

1.1.3. MYRJ59—Polyoxyethylene 100 stearate from Sigma (lot#082H0728),

1.1.4. MYRJ45—Polyoxyethylene 8 stearate from Sigma (lot#082H0304),

1.1.5. MYRJ52—Polyoxyethylene 40 stearate from Sigma (lot#104K0165),

1.1.6. Tween 80® (Polyoxyethelene 20 sorbitan monosterate) from Spectrum (lot#RT0152)

1.1.7. PEG3350 from Sigma (lot#093K0153)

1.1.8. PEG6000 from Serva (lot#15868)

1.1.9. PEG4000 from Serva (lot#17206)

3.1. Solubility of DTSI in Various Hot-Melt Formulations

The solubility of the API in one gram of solid excipients was determined by melting the solid excipients in a water bath along with the API. Additional amounts of API were added until no more could be dissolved. Dissolution or dispersion was determined by adding around 20 mg of the solid formulation to 75 mL of water in a vessel fitted with a magnetic stirrer and the time until all the solid had dissolved was measured. This information is shown in Table 4 and is listed as “dissolution behavior.”

TABLE 4
Summary of Solubility Data and Formulation Dissolution Characteristics

| | | Conc. of API^{(a)} | Bath | |
|---|---|---|---|---|
| | | (mg)/(g) | Temp. | Dissolution |
| No. | Excipients (ratio, wt/wt) | Excipients | (° C.) | Behavior |
| 1 | TPGS | 60 | 65 | Slow >15 min. |
| 2 | TPGS/Tween 80 ® (80/20) | 60 | 65 | Slow medium ~15 min. |
| 3 | TPGS/MYRJ59/Tween 80 ® | 80 | 65 | Medium <15 min. |
| | (50/30/20) | | | |
| 4* | TPGS/MYRJ59/Tween 80 ® | 90 | 65 | Medium <15 min. |
| | (40/40/20 | | | |
| 5 | PEG3350/Tween 80 ® (75/25) | 35 | 65 | Fast <5 min. |
| 6* | TPGS/PEG3350/Tween 80 ® | 70 | 65 | Fast <5 min. |
| | (40/40/20) | | | |
| 7* | TPGS/MYRJ45/Tween 80 ® | 70 | 65 | Slow >15 min. |
| | (40/40/20) | | | |
| 8* | MYRJ59/Labrasol ®/Tween 80 ® | 75 | 65 | Very fast <3 min. |
| | (40/40/20) | | | |
| 9* | TPGS/PEG3350 (50/50) | 70 | 65 | Slow >15 min. |
| 10* | TPGS/PEG3350/Tween 80 ® | 70 | 65 | Slow-medium ~15 min. |
| | (50/40/10) | | | |
| 11 | TPGS/MYRJ52/Tween 80 ® | 150 | 85-90 | Medium <15 min. |
| | (40/40/20) | | | |
| 12* | TPGS/MYRJ52/Tween 80 ® | 130 | 85-90 | Slow-medium ~15 min. |
| | (50/30/20) | | | |
| 13* | TPGS/MYRJ52/Tween 80 ® | 120 | 85-90 | Slow >15 min. |
| | (60/20/20) | | | |
| 14 | PEG4000/MYRJ52/Tween 80 ® | 100 | 85-90 | Fast <5 min. |
| | (40/40/20) | | | |
| 15* | PEG6000/MYRJ52/Tween 80 ® | 100 | 85-90 | Medium <15 min. |
| | (40/40/20) | | | |
| 16* | TPGS/MYRJ52/Tween 80 ® | 120 | 85-90 | Slow >15 min. |
| | (70/20/10) | | | |
| 17* | TPGS/MYRJ52/Tween 80 ® | 120 | 85-90 | Slow >15 min. |
| | (75/20/5) | | | |
| 18* | TPGS/MYRJ52 (75/25) | 130 | 85-90 | Slow >15 min. |
| 19* | TPGS/PEG3350 (75/25) | 120 | 85-90 | Slow >15 min. |
| 20* | MYRJ59/PEG3350/Tween 80 ® | 75 | 85-90 | Fast <5 min. |
| | (40/40/20) | | | |
| 21 | TPGS/PEG3350 (50/50) Un- | 60 | 85-90 | Slow >15 min. |
| | micronized | | | |
| 22 | TPGS/PEG3350 (75/25) Un- | 70 | 85-90 | Slow >15 min. |
| | micronized | | | |

*These formulations were used for obtaining Rat pharmacokinetic parameters
^{(a)}For all of the data shown in Table 4, micronized API sample was utilized.

As shown in Table 4, for a majority of formulations the dissolution of API to provide homogenous solution was achieved around 65° C. and these typically provided API solubility of ≦70mg/g. However, for several excipient combinations, in particular those that contained MYRJ or TPGS as one of the excipients, higher concentration of dissolved API could be achieved by using a higher bath temperature of up to 90° C. and using sonication. Because some of these provide super-saturated solutions, concentrations above 100 mg/g do not appear advantageous for formulations presented in Table 4.

3.2. Rat Pharmacokinetic Data for Hot-Melt Formulations

A number of formulations were packaged in capsules (Torpac size 9E), and were dosed orally to Sprague-Dawley rats (n=5). Each rat was administered a single capsule providing 10 m/kg equivalent of API. Plasma samples were collected for up to 6 hr (individual time points: 0.25, 0.5, 1, 2, 4 and 6 hr) and the amounts of API were determined using LC/MS/MS. The pharmacokinetic parameters were calculated using WinNoLin.

The summary of the key pharmacokinetic parameters (relative to micronised powder) for various formulation evaluated in rat, is shown in Table 5. This data includes pharmacokinetic parameters for neat micronized powder API that was also dosed as a single capsule per rat at 10 mg/kg, for comparison.

TABLE 5
PK Parameters: Cₘₐₓ and AUClast after 10 mg/kg Oral Dose of API,
Dosed as a Single Capsule in the Formulation Listed Relative to
Micromnized Powder, also dosed at 10 mg/kg to Sprague-Dawley Rats.

| | Cₘₐₓ Ratio vs | AUC Ratio vs |
|---|---|---|
| Formulation | Micronized | Micronized |
| Micronized powder | 1.00 | 1.00 |
| MYRJ59/Labrasol ®/Tween 80 ® | 0.84 | 0.43 |
| (4/4/2) | | |
| TPGS/MYRJ52/Tween 80 ® | 1.85 | 1.49 |
| (4/4/2) | | |
| MYRJ 52/TPGS/Tween 80 ® | 2.10 | 1.84 |
| (3/5/2) | | |
| TPGS/MYRJ52/Tween 80 ® | 1.45 | 1.19 |
| (6/2/2) | | |
| MYRJ 52/TPGS/Tween 80 ® | 1.40 | 1.15 |
| (2/7/1) | | |
| MYRJ 52/TPGS/Tween 80 ® | 1.53 | 1.28 |
| (20/75/5) | | |
| MYRJ 52/TPGS (25/75) | 1.87 | 1.45 |
| MYRJ59/PEG 3350/Tween 80 ® | 0.80 | 0.70 |
| (4/4/2) | | |
| MYRJ59/TPGS/Tween 80 ® | 2.69 | 1.00 |
| (4/2/2) | | |
| TPGS/PEG3350/Tween 80 ® | 1.54 | 1.14 |
| (4/4/2) | | |
| TPGS/PEG 3350/Tween 80 ® | 3.64 | 2.29 |
| (5/4/1) | | |
| TPGS/PEG 3350 (1/1) | 3.34 | 2.30 |
| PEG 3350/TPGS (25/75) | 3.33 | 2.25 |
| PEG 6000/TPGS/Tween 80 ® | 3.14 | 2.04 |
| (4/4/2) | | |

3.3. Dog Pharmacokinetic Data for Hot-Melt Formulations

Two TPGS/PEG3350 formulations [TPGS/PEG 3350 (1/1) and PEG 3350/TPGS (25/75)] were subsequently dosed orally to dogs at 5 mg/kg and 30 mg/kg for the determination of pharmacokinetic parameters. Three dogs were dosed for each dose group with each of the formulations and with 5 mg/kg and 30 mg/kg dose of micronized powder in capsules for comparative PK analysis. These data showed that while the two hot-melt formulations and micronized powder dosed to dog provided similar AUC, the two hot-melt formulations provided significantly higher Cₘₐₓ, as shown in Table 6.

TABLE 6
Relative improvement in Cₘₐₓ (average of n = 3 for each
formulations) following oral dosing of these formulations to dogs.

| | | TPGS- | TPGS- |
|---|---|---|---|
| Dose | | PEG3350 | PEG3350 |
| (mpk) | Micronized | 1:1 | 3:1 |
| 5 | 1.00 | 2.84 | 2.85 |
| 30 | 1.00 | 1.98 | 2.06 |

3.4 Example of Hot-melt Capsule Preparation

The following formulation parameters were used in an amount sufficient to prepare sixty thousand hot-melt capsules having 100 mg dosage strength.

| | | Weight/ |
|---|---|---|
| Component | Quantity | Weight % |
| DTSI | 6.001 kg | 12.00 |
| Polyethylene Glycol 3350, NF, Ph.Eur Powder | 10.99 kg | 21.99 |
| Vitamin E Polyethylene Glycol Succinate, NF | 33.01 kg | 66.01 |

Polyethylene Glycol 3350 and Vitamin E Polyethylene Glycol 3350 were heated and stirred to 90±5° C. in a kettle until melted. DTSI was added to the molten mixture and stirred until dissolved. The temperature of the mixture was then reduced to 70±5° C. and the cooled mixture was filled into size 00 hard gelatin capsules using standard liquid capsule filler such as the Shionogi Capsule Liquid Filler. The capsules were banded to prevent leaks with a standard capsule machine such as the Shionogi Hard Capsule Sealing Machine using a banding solution containing 1 part Polysorbate 80, NF, 23.5 parts Gelatin 220 LB bloom HC Grade USP and 88 parts USP Purified Water where the average capsule weight gain during the banding process is 8.3 mg after most of the water is dried off.

3.5. Additional Formulations.

Initial studies indicate that hot-melt formulations that include small amounts of a polymer such as hydroxypropyl methylcellulose (HPMC) allow preparation of hot soluble formulations which, upon cooling, can provide homogeneous solid formulations with significantly high API load per gram of the formulated solid. One such solid formulation contains a suspension of API and Vitamin E TPGS (50/50 mixture of API in Vitamin E TPGS) and a small amount of HPMC. Under visual observations, this forms a uniform solution that solidifies uniformly.

In another embodiment, the formulation of the invention contains clear solution of (20% by wt.) DTSI, (10%) TPGS and (10%) poly (ethylene oxide) (Polyox™) in water, lyophilized to provide a solid dosage. In yet another embodiment, the formulation contains, when hot, clear solution of (20-40% by wt.) DTSI, (15-30%) PEG3350, (15-30%) Tween80 and (5-15%) diethanolamine, which, upon cooling, provides solid formulation with significantly high API load per gram of the formulated solid.

The contents of each of the references cited herein, including the contents of the references cited within the primary references, are herein incorporated by reference in their entirety.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

We claim:

1. A solid, single-phase pharmaceutical composition comprising DTSI 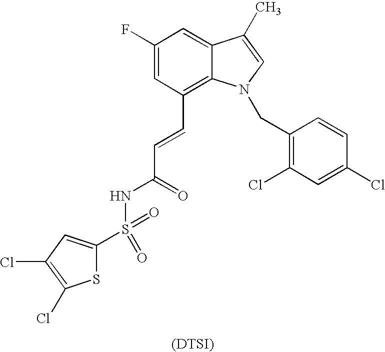
or a pharmaceutically acceptable salt thereof, and at least one pharmaceutically acceptable excipient.

2. A composition according to claim 1, wherein the at least one pharmaceutically acceptable excipient is chosen from Vitamin E TPGS, polyethylene glycol, and combinations thereof.

3. A composition according to claim 1, wherein the at least one pharmaceutically acceptable excipient is chosen from Vitamin E TPGS, polyethylene glycol, hydroxypropyl methylcellulose, and combinations thereof.

4. A composition according to claim 1, wherein the at least one pharmaceutically acceptable excipient is chosen from Vitamin E TPGS, polyethylene glycol, hydroxypropyl methylcellulose, polyglycolyzed glyceride, polyoxyethylene glycol ester, polyoxyethylene sorbitan fatty acid ester, choline, and combinations thereof.

5. A composition according to claim 4, wherein said polyglycolyzed glyceride is a glyceryl caprylate/caprate and polyethylene glycol caprylate/caprate complex.

6. A composition according to claim 4, wherein the polyoxyethylene glycol ester is chosen from poloxyethylene 8 stearate, poloxyethylene 40 stearate, polyoxyethylene 100 stearate, and combinations thereof.

7. A composition according to claim 4, wherein the polyoxyethylene sorbitan fatty acid ester is polyoxyethylene 20 sorbitan monooleate.

8. A composition according to claim 1, wherein the pharmaceutically acceptable salt is chosen from a salt with a pharmaceutically acceptable primary, secondary, or tertiary amine compound, and a pharmaceutically acceptable quaternary ammonium compound.

9. A composition according to claim 8, wherein the pharmaceutically acceptable salt is chosen from a salt with lysine, arginine, betaine, sarcosine, choline, choline phosphate, tromethamine, ethanolamine, diethanolamine, and triethanolamine.

10. A composition according to claim 1 wherein the at least one pharmaceutically acceptable excipient is a P-glycoprotein inhibitor.

11. A composition according to claim 10 wherein the P-glycoprotein inhibitor is chosen from polyoxyethylene 20 sorbitan monooleate, polyoxyl 35 castor oil, polyoxyl 40 castor oil, Vitamin E TPGS, and combinations thereof.

12. A composition according to claim 1 wherein the at least one pharmaceutically acceptable excipient is chosen from Vitamin E TPGS, polyethylene glycol, hydroxypropyl methylcellulose, a P-glycoprotein inhibitor, and combinations thereof.

13. A composition according to claim 12, wherein the P-glycoprotein inhibitor is chosen from Vitamin E TPGS, polyoxyethylene 20 sorbitan monooleate, polyoxyl 35 castor oil, polyoxyl 40 castor oil, and combinations thereof.

14. A composition according to claim 1, wherein the at least one pharmaceutically acceptable excipient is chosen from a combination of Vitamin E TPGS and polyethylene glycol, and wherein a weight by weight ratio of Vitamin E TPGS to polyethylene glycol is about 1:1.

15. A composition according to claim 1, wherein the at least one pharmaceutically acceptable excipient is chosen from a combination of Vitamin E TPGS and polyethylene glycol, and wherein a weight by weight ratio of Vitamin E TPGS to polyethylene glycol is about 3:1.

16. A composition according to claim 1, wherein a weight by weight ratio of DTSI, or pharmaceutically acceptable salt thereof, to the at least one pharmaceutically acceptable excipient is in a range from about 1:100 to about 1:1.

17. A composition according to claim 1, wherein a weight by weight ratio of DTSI, or pharmaceutically acceptable salt thereof, to the at least one pharmaceutically acceptable excipient is in a range from about 1:20 to about 1:1.

18. A composition according to claim 1, wherein a weight by weight ratio of DTSI, or pharmaceutically acceptable salt thereof, to the at least one pharmaceutically acceptable excipient is in a range from about 1:15 to about 1:5.

19. A composition according to claim 1 further comprising one or more therapeutic agents chosen from a platelet aggregation inhibitor, an HMG-CoA reductase inhibitor, an antihyperlipidemic agent and a cyclooxygenase inhibitor.

20. A composition according to claim 19, wherein said platelet aggregation inhibitor is chosen from tirofiban, dipyridamole, clopidogrel and ticlopidine.

21. A composition according to claim 19, wherein said HMG-CoA reductase inhibitor is chosen from lovastatin, simvastatin, pravastatin, rosuvastatin, mevastatin, atorvastatin, cerivastatin, pitavastatin, and fluvastatin.

22. A composition according to claim 19, wherein said cyclooxygenase inhibitor is chosen from rofecoxib, meloxicam, celecoxib, etoricoxib, lumiracoxib, valdecoxib, parecoxib, cimicoxib, diclofenac, sulindac, etodolac, ketoralac, ketoprofen, piroxicam, and LAS-34475.

23. A composition according to claim 1, wherein the composition is a capsule, troche, dispersion, suspension, solution, patch, or a tablet.

24. A method for the treatment or prophylaxis of a prostaglandin-mediated disease or condition comprising administering to a mammal in need thereof a therapeutically effective amount of a composition according to claim 1.

25. The method of claim 24, wherein said disease or condition is chosen from pain, fever or inflammation associated with rheumatic fever, influenza or other viral infections, common cold, low back and neck pain, skeletal pain, post-partum pain, dysmenorrhea, headache, migraine, toothache, sprains and strains, myositis, neuralgia, synovitis, arthritis, including rheumatoid arthritis, degenerative joint diseases, gout and ankylosing spondylitis, bursitis, burns including radiation and corrosive chemical injuries, sunburns, pain following surgical and dental procedures, immune and autoimmune diseases; cellular neoplastic transformations or metastatic tumor growth; diabetic retinopathy, tumor angiogenesis; prostanoid-induced smooth muscle contraction associated with dysmenorrhea, premature labor, asthma or eosinophil related disorders; Alzheimer's disease; glaucoma; bone loss; osteoporosis; Paget's disease; peptic ulcers, gastritis, regional enteritis, ulcerative colitis, diverticulitis or other gastrointestinal lesions, GI bleeding; coagulation disorders selected from hypoprothrombinemia, hemophilia and other bleeding problems; kidney disease; thrombosis, myocardial infarction, stroke; and occlusive vascular disease.

26. The method of claim 25, wherein said disease is occlusive vascular disease.

27. A method for reducing plaque in the treatment of atherosclerosis comprising administering to a mammal in need thereof a therapeutically effective amount of a composition according to claim 1.

28. A method for the promotion of bone formation or for cytoprotection comprising administering to a mammal in need thereof a therapeutically effective amount of a composition according to claim 1.

29. A method for the treatment or prophylaxis of pain, inflammation, atherosclerosis, myocardial infarction, stroke or vascular occlusive disorder comprising administering to a mammal in need thereof a therapeutically effective amount of a composition according to claim 1.

30. A process for preparation of a solid oral pharmaceutical composition in unit dosage form, said process comprising
a) mixing DTSI 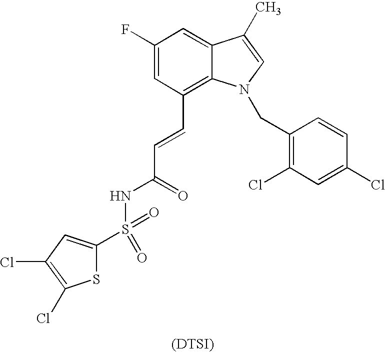
or a pharmaceutically acceptable salt thereof, at least one pharmaceutically acceptable fusible excipient, and, optionally, at least one pharmaceutically acceptable excipient;
b) subjecting the mixture to injection molding or extrusion; and
c) processing the mixture into said dosage form.

31. The process of claim 30, wherein said mixing step is performed at a temperature ranging from about 5 to about 15 degrees C. higher than a melting temperature of the at least one pharmaceutically acceptable fusible excipient, and wherein if more than one pharmaceutically acceptable fusible excipients having different melting points are used, said mixing is carried out at a temperature ranging from about 5 to about 15 degrees C. higher than a melting temperature of a pharmaceutically acceptable fusible excipient with a highest melting point.

32. The process of claim 31, wherein a weight by weight ratio of DTSI, or pharmaceutically acceptable salt thereof, to the at least one pharmaceutically acceptable fusible excipient is in a range from about 1:15 to about 1:5.

33. The process of claim 30, wherein the at least one pharmaceutically acceptable fusible excipient is chosen from Vitamin E TPGS, polyethylene glycol, and combinations thereof.

34. The process of claim 30, wherein the at least one pharmaceutically acceptable fusible excipient is chosen from Vitamin E TPGS, polyethylene glycol, hydroxypropyl methylcellulose, and combinations thereof.

35. The process of claim 30, wherein the at least one pharmaceutically acceptable fusible excipient is chosen from Vitamin E TPGS, polyethylene glycol, hydroxypropyl methylcellulose, a P-glycoprotein inhibitor, and combinations thereof.

36. The process of claim 35, wherein the P-glycoprotein inhibitor is Vitamin E TPGS, polyoxyethylene 20 sorbitan monooleate, or combinations thereof.

37. The process of claim 30, wherein the step of subjecting the mixture to injection molding or extrusion results in a solid, single-phase composition.

38. The process of claim 30, wherein the at least one pharmaceutically acceptable excipient is choline.

39. A pharmaceutical composition comprising DTSI 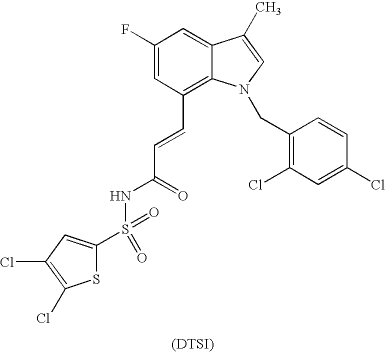
or a pharmaceutically acceptable salt thereof, and at least one pharmaceutically acceptable excipient, wherein DTSI is present as particles having size of about 1 nm to about 1000 nm.

40. A pharmaceutical composition according to claim 39, wherein the pharmaceutically acceptable salt is chosen from a salt with a pharmaceutically acceptable primary, secondary, or tertiary amine compound, and a pharmaceutically acceptable quaternary ammonium compound.

41. A pharmaceutical composition according to claim 40, wherein the pharmaceutically acceptable salt is chosen from a salt with lysine, agrinine, betaine, sarcosine, choline, choline phosphate, tromethamine, ethanolamine, diethanolamine, and triethanolamine.

42. A pharmaceutical composition according to claim 39, wherein the composition is a capsule, troche, dispersion, suspension, solution, patch, or a tablet.

43. A pharmaceutical composition according to claim 39, wherein the at least one pharmaceutically acceptable excipient is choline.

44. A pharmaceutical composition comprising a pharmaceutically acceptable salt of DTSI 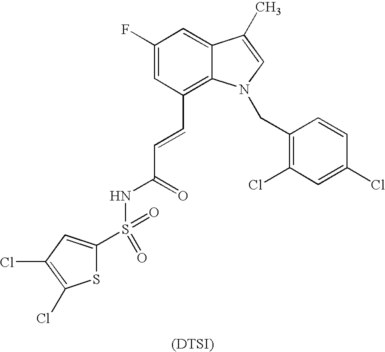
and at least one pharmaceutically acceptable excipient, wherein the pharmaceutically acceptable salt is chosen from a salt with a pharmaceutically acceptable primary, secondary, or tertiary amine compound, and a pharmaceutically acceptable quaternary ammonium compound.

45. A pharmaceutical composition according to claim 44, wherein the pharmaceutically acceptable salt is chosen from a salt with choline, choline phosphate, tromethamine, ethanolamine, diethanolamine, and triethanolamine.

46. A pharmaceutical composition according to claim 44, wherein the composition is a capsule, troche, dispersion, suspension, solution, patch, or a tablet.

47. A pharmaceutical composition according to claim 44, wherein the at least one pharmaceutically acceptable excipient is choline.
